(19) United States
(12) Patent Application Publication
(10) Pub. No.: US 2026/0294576 A1
(43) Pub. Date: Oct. 1, 2026

(54) SYSTEMS INCLUDING A SURGICAL TRAY FOR ADJUSTABLE INSTRUMENT POSITIONING

(71) Applicant: Alcon Inc., Fribourg (CH)

(72) Inventor: Robert Y. Diehl, Dana Point, CA (US)

(21) Appl. No.: 19/563,210

(22) Filed: Mar. 11, 2026

Related U.S. Application Data
(60) Provisional application No. 63/778,538, filed on Mar. 27, 2025.

(51) Int. Cl. A61B 50/33

(52) U.S. Cl. CPC A61B 50/33

(57) ABSTRACT

A surgical tray system comprising a tray comprising a track positioned on an instrument surface of the tray (e.g., along a perimeter), and a clip configured to be adjustably coupled to and positioned along the track. The clip is further configured to couple to a cable of a surgical instrument and secure the cable on the tray.

### INTRODUCTION

In many types of medical settings, a healthcare professional will use an array of instruments to perform delicate procedures on a patient. These instruments, together with certain supplies and consumables, are typically placed on a tray located both near the patient and within easy reach of the healthcare professional. Most people who have ever visited a dentist are familiar with the tray used by a dental hygienist or a dentist when performing either cleaning or more complex dental procedures on their teeth. The tray is positioned near the patient’s head so that those items needed to complete the dental procedure are in easy reach of the healthcare professional.

Trays similar to those used by dentists are also used in surgical settings, including operating rooms for ophthalmic surgery. During an ophthalmic surgical procedure, a surgeon is typically positioned near/over the head of the patient to enable easy access to the patient’s eyes. The surgeon may use a variety of different instruments, supplies, and consumables during the procedure. These instruments, supplies, and consumables are typically placed within easy reach of the surgeon on a tray that may be further coupled to a surgical console.

During certain ophthalmic surgical procedures, the instruments placed on the tray include instruments with a wired connection to the surgical console. And, depending on the location of the surgical tray relative to the surgical console and/or surgeon, such instruments may need to be particularly oriented and/or repositioned to ensure that wiring and/or tubing associated with the instruments is kept clear of the surgical area. This can lead to inefficiencies and/or inconvenience for the surgeon when performing the ophthalmic surgical procedure.

### SUMMARY

The present disclosure generally relates to systems and apparatuses for ophthalmic surgical procedures.

Certain embodiments of the present disclosure provide a surgical tray system. The surgical tray system comprises a tray comprising a track positioned on an instrument surface (e.g., along a perimeter) of the tray, and a clip configured to be adjustably coupled to and positioned along the track. The clip is further configured to couple to a cable of a surgical instrument and secure the cable on the tray.

Certain embodiments of the present disclosure provide a surgical tray system. The surgical tray system comprises a tray comprising an aperture positioned on an instrument surface (e.g., along a perimeter) of the tray, and a clip configured to be positioned within the aperture. The clip is further configured to couple to a cable of a surgical instrument and secure the cable on the tray.

The following description and the related drawings set forth herein detail certain illustrative features of one or more embodiments, including those described above.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a topside isometric view of an example surgical tray, according to embodiments described herein.

FIG. 2 illustrates a topside isometric view of an example surgical tray with tracks for securing one or more cables thereon, according to embodiments described herein.

FIG. 3A illustrates a magnified view of a track of FIG. 2, according to embodiments described herein.

FIG. 3B illustrates a magnified view of another exemplary type of track, according to embodiments described herein.

FIG. 3C illustrates a magnified view of another exemplary type of track, according to embodiments described herein.

FIG. 4 illustrates a perspective view of an example clip for securing one or more cables onto a track of a surgical tray, according to embodiments described herein.

FIG. 5 illustrates a topside isometric view of an example surgical tray having individual apertures for attaching clips thereon, according to embodiments described herein.

FIG. 6 illustrates a perspective view of an example clip configured to couple to an aperture of a surgical tray for securing one or more cables thereon, according to embodiments described herein.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure, but is merely representative of various embodiments. While the various aspects of the embodiments are presented in the Figures, the Figures are not necessarily drawn to scale unless specifically indicated.

Note that, as described herein, a “distal” end, side, or portion of a component refers to the end, side, or the portion that is closer to the patient’s body during the use thereof. On the other hand, a “proximal” end, side, or portion of the component refers to the end, side, or portion that is distanced further away from the patient’s body.

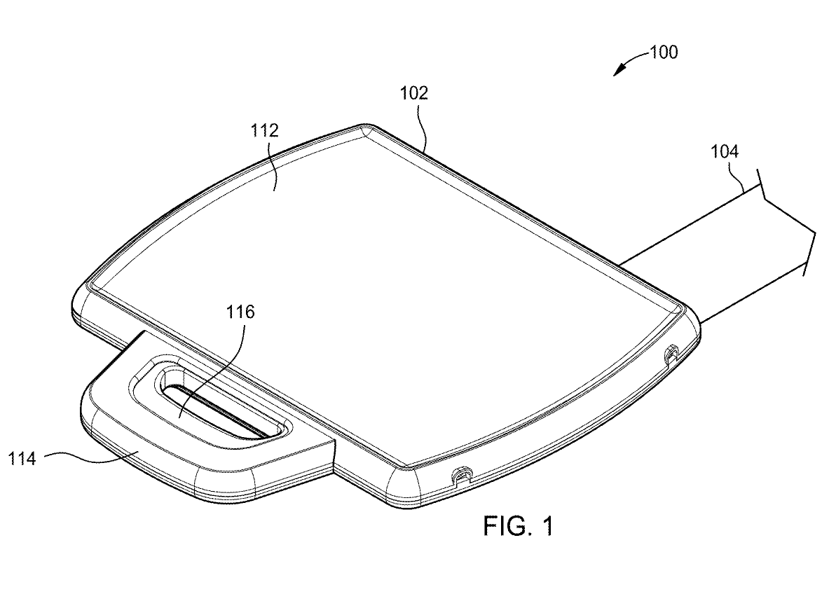

FIG. 1 illustrates a topside isometric view of an example surgical tray assembly 100, according to embodiments described herein. The surgical tray assembly 100 can, in certain embodiments, be an integral part of a surgical console. In certain other embodiments, the surgical tray assembly 100 is removably coupled to a surgical console. As shown, surgical tray assembly 100 comprises an arm 104, a tray 102, a tray positioning handle 114, and a release handle 116.

The arm 104, in certain embodiments, includes or is attached to one or more adjustable joints and/or mounts for coupling the tray 102 to the surgical console. For example, in certain embodiments, the arm 104 is coupled to a tray mount disposed on a bottom or lateral side of the tray 102. The one or more adjustable joints and/or mounts allow the tray 102 and/or the arm 104 to be positioned (e.g., moved or rotated) with respect to each other and/or the surgical console. In certain embodiments, the tray 102 and/or arm 104 can be releasably locked in place once adjusted to a desired position. For example, in such embodiments, actuation (e.g., pressing and/or pulling) of the release handle 116 by a user can release, or unlock, the arm 104 and/or tray 102 for positional adjustment; once the arm 104 and/or tray 102 is adjusted to a desired position, the user can release the release handle 116 to lock the arm 104 and/or tray 102 in place.

The surgical tray assembly 100 described herein is entirely mechanical, however, those of ordinary skill in the art will understand that in an alternative embodiment, one or more powered actuators (e.g., electromechanical, pneumatic, etc.) can be used to move the components, including the arm 104 and joints of the surgical tray assembly 100.

The tray 102 includes an instrument surface 112 with the capacity to hold instruments, equipment, supplies, and any consumables (altogether referred to herein as “instruments” or “surgical instruments”) needed during a surgical procedure, in addition to any associated cables and/or tubes (altogether referred to herein as “cables”). While the tray 102 can be moved and/or rotated with respect to the surgical console to be within easy reach of a surgeon, in certain cases, the surgeon desires or the surgical procedure necessitates that certain instruments be secured in specific, fixed positions on the tray 102, including instruments that are wiredly connected to the surgical console.

During surgical procedures, the tray 102 is typically covered with a transparent or translucent surgical drape and/or tray cover to maintain a sterile surgical field and prevent contamination by tray 102. Instruments are placed on top of the surgical drape on the instrument surface 112 and/or can be secured at fixed locations along a perimeter of the tray 102. However, conventional trays do not provide any efficient means of securing the instruments onto the instrument surface 112, and/or do not facilitate customizable arrangement of the instruments on the instrument surface 112 based on the surgeon’s preferred configuration and/or the type of surgical procedure. Particularly, current trays have limited, fixed options for cable management and/or placement of instruments, thereby not allowing surgeons to secure instruments and their cables in desired configurations. This inability to customize the arrangement of the surgical instruments on the tray results in inefficiencies in the surgical procedure and/or inconvenience to the surgeon during the surgical procedure.

The embodiments described herein provide tray systems that include one or more components for securing surgical instruments and/or managing cables on a surgical tray for use during surgical procedures, such as ophthalmic surgical procedures. In certain embodiments, the tray systems include one or more tracks arranged along a perimeter of an instrument surface of a tray, wherein such tracks are configured to receive one or more removable clips for securing one or more cables (e.g., instrument cables) to the tray. In certain embodiments, the tray systems include one or more apertures disposed along the perimeter of the instrument surface of the tray, wherein such apertures are configured to receive one or more removable clips for securing one or more cables to the tray.

The tracks and/or apertures of the embodiments described herein allow versatility in tray arrangement so that a surgeon can position and/or secure one or more instruments in a desired configuration on the tray for a given procedure. Furthermore, while the tracks and/or apertures provide flexibility in positioning the instruments, they also prevent, or at least reduce, the likelihood of the instruments undesirably sliding along the tray when the tray or its surgical drape is adjusted or moved. And, after use, the tray may be reconfigured by relocating one or more removable clips to accommodate a desired configuration for another type of procedure, and/or according to a desired configuration of another surgeon using the surgical system.

Accordingly, the tray systems described herein provide improved customization of the surgical environment based on the surgeon’s preference and/or based on the type of procedure to be performed. Additionally, such tray systems facilitate the placement of cables of one or more instruments in an organized manner to prevent cable interference in the surgical area and/or to prevent movement of one or more instruments on the tray. Thus, the surgeon is able to manipulate the placement of instruments on the tray based on the surgeon’s preference, and the location of the instruments on the tray can be maintained throughout the duration of the surgical procedure, without the worry that one or more instruments may unintentionally slip off of the tray.

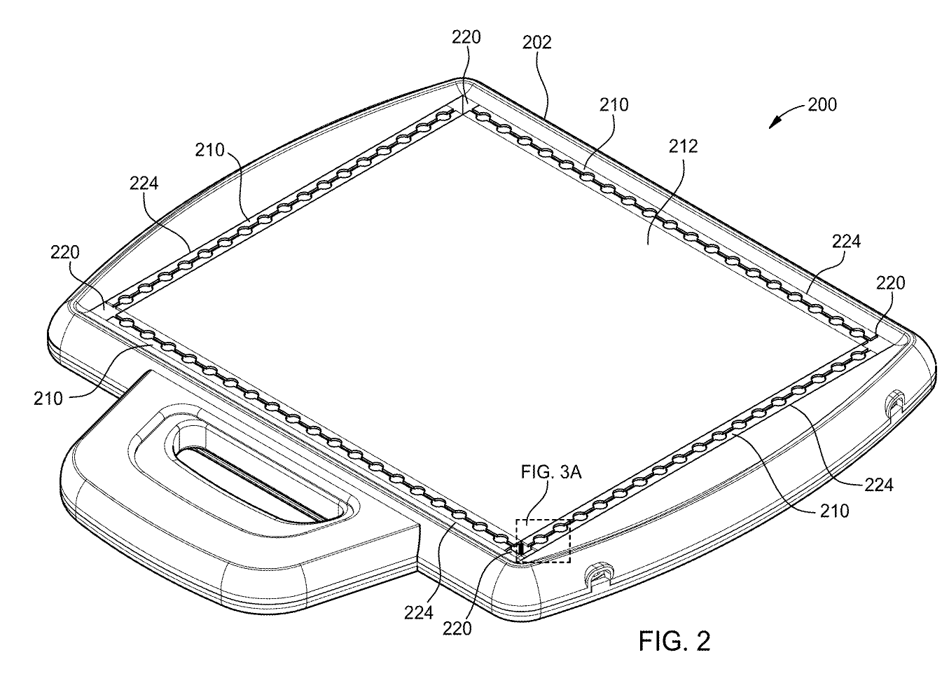

Example embodiments of the tray systems are described in further detail with reference to FIGS. 2-6.

FIG. 2 illustrates a topside isometric view of an example surgical tray assembly 200 having tracks 210, according to embodiments described herein. As shown, a number of tracks 210 are disposed along a perimeter 224 of an instrument surface 212 of the tray 202. Note that although the tracks illustrated in FIG. 2 are all labeled as tracks 210, each track or pair of tracks can have a different length. For example, in FIG. 2, there are two pairs of tracks on opposite sides of the rectangular perimeter 224, with each pair being equal in length but different from the length of the other pair of tracks because of the rectangular shape of the instrument surface 212. However, if the instrument surface 212 is square shaped, then all tracks 210 can have equal lengths.

Although the tracks 210 shown in FIG. 2 include four separate tracks 210 positioned along four sides of the perimeter 224 of the instrument surface 212, the surgical tray assembly 200 can include any number of separate tracks 210 (e.g., 1, 2, 3, 5, 6, etc.). For example, in certain embodiments, a single continuous track 210 may be disposed around the entire perimeter 224 of the instrument surface 212 (e.g., disposed parallel to all four edges of the instrument surface 212 in FIG. 2). In certain other embodiments, the tracks 210 are positioned along less than four sides of the perimeter 224 of the instrument surface 212. For example, tracks 210 can be positioned around a single side, two sides, or three sides of the perimeter 224 of the instrument surface 212. In certain embodiments, open ends 220 of one or more tracks 210 are exposed through the instrument surface 212 of the tray 202.

In certain embodiments, the tracks 210 are formed of a metallic material, such as titanium, aluminum, stainless steel, other alloys, or the like. In certain embodiments, the tracks 210 are formed of a thermoplastic material, such as high-density polyethylene (HDPE). However, the tracks 210 can be formed of other metal and/or plastic materials instead of or in addition to those described herein.

As shown in FIG. 2, in certain embodiments, the tracks 210 are secured onto the instrument surface 212 of the tray 202. However, in certain other embodiments, the tracks 210 are secured to side walls of the tray 202, or a bottom surface of the tray 202. In certain embodiments, the tracks 210 are secured to the tray 202 using one or more fasteners, such as screws, bolts, or the like. In certain embodiments, the tracks 210 are molded to the tray 202 or otherwise secured to the tray 202 via interference fastening, mechanical interlocking joints, welding, adhesives, etc.

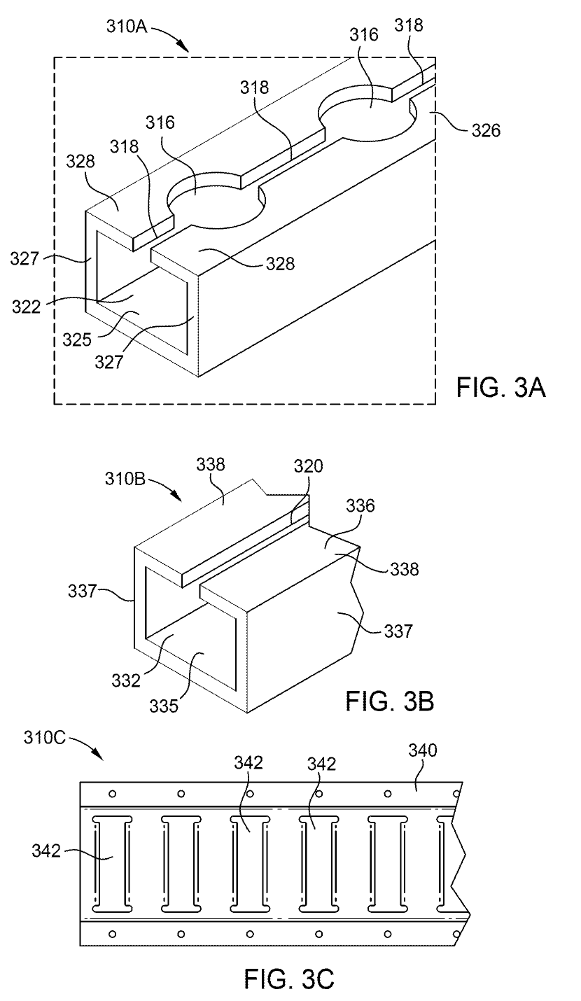

FIG. 3A illustrates a magnified view of a portion of an exemplary type of track 210 of FIG. 2, according to embodiments described herein. For example, FIG. 3A illustrates an L-track 310A, which includes one or more rails 326 having a T-shaped axial profile formed by extrusion or a similar manufacturing process.

In the example of FIG. 3A, the L-track 310A includes a singular rail 326 having two sidewalls 327 extending perpendicularly from a base 325 and running substantially parallel to one another. A lip 328 extends inwardly from each sidewall 327 at an end of the sidewall 327 opposite the base 325. Altogether, the sidewalls 327, the lips 328, and the base 325 at least partially define an interior portion 322. The two lips 328 further form a linear channel 318 disposed above the interior portion 322. The channel 318 and the interior portion 322 extend along a length of the L-track 310A. A series of apertures 316 is formed (e.g., via machining such as drilling or stamping) along the channel 318, wherein each aperture 316 is spaced apart from adjacent aperture(s) 316 via a length of the channel 318. In other words, the channel 318 connects adjacent apertures 316. Generally, the apertures 316 can be circular in shape but other shapes are also contemplated.

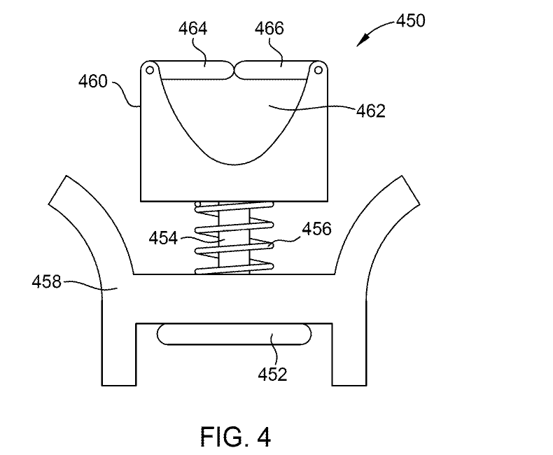

The L-track 310A is configured to receive a clip, such as the clip described in relation to FIG. 4. The clip can be secured within the L-track 310A by inserting a base of the clip through an aperture 316 and into the interior portion 322 and thereafter translating the clip through the channel 318 and apertures 316 to a desired position along the L-track 310A. In such examples, the clip is configured to be secured along the channel 318 of the L-track 310A.

FIG. 3B illustrates a magnified view of a portion of another exemplary type of track 210 of FIG. 2, according to embodiments described herein. For example, FIG. 3B illustrates a T-track 310B, which includes one or more rails 336 having a T-shaped axial profile formed by extrusion or similar manufacturing process.

The illustrated T-track 310B is similar to the L-track 310A in FIG. 3A and includes a singular rail 336 having two sidewalls 337 extending perpendicularly from a base 335 and running substantially parallel to one another. A lip 338 extends inwardly from each sidewall 337 at an end of the sidewall 337 opposite the base 335. Altogether, the sidewalls 337, the lips 338, and the base 335 at least partially define an interior portion 332. The two lips 338 further form a linear channel 320 extending along a length of the T-track 310B. However, unlike the L-track 310A, there are no apertures formed along the channel 320 of the T-track 310B. Thus, in such embodiments, a clip can be secured to the T-track 310B by inserting a base of the clip into the interior portion 332 via open ends 220 of the track(s) 210 and then sliding the clip along the channel 320. In some examples, the open ends 220 are disposed at or near one or more corners of the tray 202; however, other positions are also contemplated for the open ends 220.

FIG. 3C illustrates a magnified view of a portion of yet another exemplary type of track 210 of FIG. 2, according to embodiments described herein. For example, FIG. 3C illustrates an E-track 310C, which includes a singular rail 340 with a plurality of slots 342 formed along a length of the E-track 310C. In such embodiments, one or more clips can be secured within the slots 342 of the E-track 310C.

In still further embodiments, the one or more tracks 210 can include magnetic strips formed of one or more surgical-grade magnetic materials. Examples of suitable materials include ferrous metals such as iron, cobalt, nickel, neodymium iron boron, and the like. In such embodiments, clips with magnetic bases can be coupled to the magnetic strips for securing one or more cables thereto.

FIG. 4 is a side view of an example clip 450 configured to be releasably attached along tracks 210 of surgical tray assembly 200 for securing one or more cables (e.g., instrument cables) thereto, according to certain embodiments described herein. Hence, clip 450 can be referred to as a “cable clip” in some examples herein. Clip 450 includes a stud 452, a pin 454, a biasing mechanism 456, a base 458, and a cable securing interface 460. A distal end of the pin 454 is attached to the stud 452, while a proximal end of the pin 454 is attached to the cable securing interface 460. The pin 454 can be welded, adhered, or otherwise secured to the stud 452 and/or the cable securing interface 460. The biasing mechanism 456 is positioned around the pin 454 between the cable securing interface 460 and the base 458. In certain embodiments, the biasing mechanism 456 includes a spring, such as a compression spring, a conical spring, or another type of spring.

In a resting state of the clip 450, as shown in FIG. 4, the biasing mechanism 456 biases the cable securing interface 460 away from the base 458, causing the stud 452 to be pressed against the base 458. A user can manually depress the cable securing interface 460 towards the base 458, causing the biasing mechanism 456 to compress and the stud 452 to move away from the base 458. With the stud 452 spaced apart from the base 458, the clip 450 can be attached to, and/or rearranged along, a track 210.

For example, with the stud 452 spaced apart from the base 458, the stud 452 can be inserted through an open end or aperture of a track 210 and then translated along the track 210 with the stud 452 disposed below the lips of the track 210 (e.g., within the track 210). When the stud 452 is disposed below the lips of the track 210, the pin 454 is disposed through the channel of the track 210, and the base 458, the biasing mechanism 456, and the cable securing interface 460 are disposed above the lips of the track 210. The user can then release the cable securing interface 460 once the clip 450 is arranged at a desired location along the track 210, causing the biasing mechanism 456 to extend and press the stud 452 up against the lips of the track 210, thereby securing the clip 450 within the track 210. The process of securing the clip 450 along the track 210 can be duplicated for any number of other clips 450. For example, if a surgeon anticipates using three instruments, the surgeon can secure three clips 450 in various positions along the one or more tracks 210 of the tray 202 to secure cables from each of the instruments. Once the desired number of clips 450 is attached to the one or more tracks 210, one or more cables can be secured to the clips 450.

Generally, clip(s) 450 can be attached to the track(s) 210 prior to the start of a surgical procedure, before the tray 202 and/or the surgical console is brought into the operating room, for example. Upon attachment of the clip(s) 450 and prior to the surgical procedure, the tray 202 can then covered with a surgical drape and/or a tray cover to facilitate a sterile surgical environment and/or prevent contamination of the tray 202 as described herein. The surgical drape and/or tray cover does not disrupt the function of the clip(s) 450 or the cable securing interface 460 described herein.

The cable securing interface 460 of the clip 450 is depicted in FIG. 4 as a generally U-shaped structure with two retaining arms 464 and 466 configured to allow one or more cables to be releasably secured within an interior portion 462 of the cable securing interface 460. However, other shapes for the cable securing interface 460, and/or other numbers of retaining arms, are also contemplated. For example, in certain embodiments, the cable securing interface 460 is generally cuboid or triangular in shape; in certain embodiments, the cable securing interface 460 includes only one retaining arm 464 or 466.

In certain embodiments, the retaining arms 464 and 466 are arranged to be substantially perpendicular to the pin 454 in a resting position. In certain embodiments, the retaining arms are configured to flex, or bend, from/to their resting position to facilitate pressing/pulling of a cable into/out from the interior portion 462. In certain other embodiments, the retaining arms 464 and 466 are spring biased and configured to swivel from/to their resting position along a hinge to facilitate pressing/pulling of a cable into/out from the interior portion 462. To secure a cable within the interior portion 462, a user (e.g., a surgeon) can press the cable into the retaining arms 464 and 466 toward the interior portion 462, causing the retaining arms 464 and 466 to flex or swivel down into the interior portion 462 and away from their resting position, allowing the cable to slide past the retaining arms 464 and 466. Once the cable is disposed in the interior portion 462 and beyond the retaining arms 464 and 466, the retaining arms 464 and 466 are configured to flex or swivel back to their resting position.

In certain embodiments, the retaining arms 464 and 466 are configured to swivel inward (toward the interior portion 462) when a force is applied to a top surface of the retaining arms 464 and 466, but not outward (away from the interior portion 462) beyond their resting position in response to a force applied to a bottom surface of the retaining arms 464 and 466. This prevents unintentional release of cables from the clip 450 as caused by a surgeon accidentally pulling upward on the cable during use. Thus, to release the cable in such embodiments, a user can press down on the retaining arms 464 and 466 to enable egress of the cable from the interior portion 462.

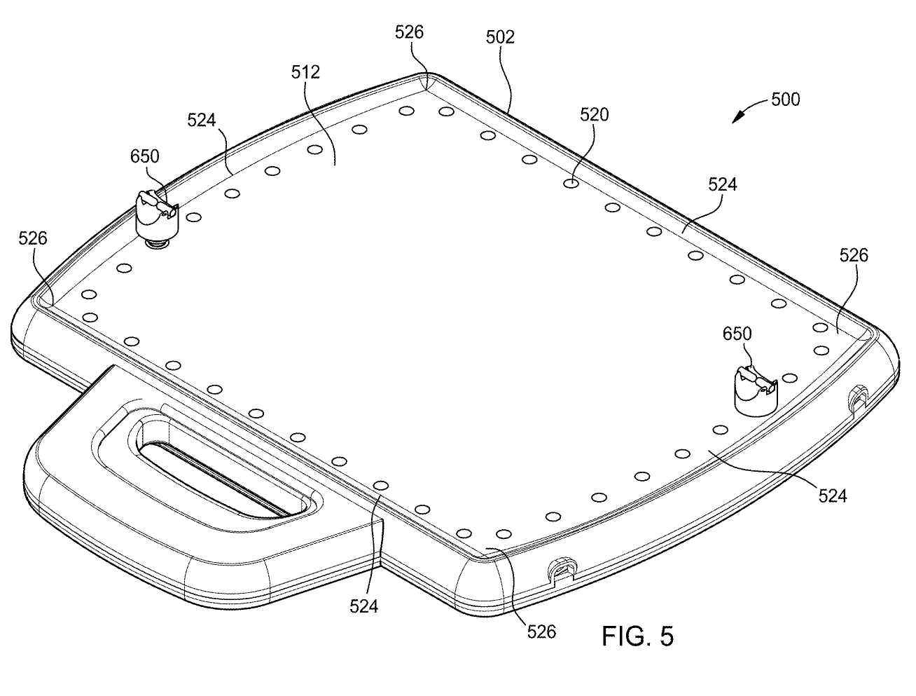

While a spring-biased clip 450 is described with reference to FIG. 4, the clip 450 can include any suitable mechanical or magnetic interface for attaching the clip 450 to a track 210. For example, instead of a stud and a biasing mechanism, the clip can include a threaded base to allow the clip to be screwed into a threaded aperture, or a cylindrical base configured to be press fit into an aperture, as described in reference to FIG. 5. Further, although shown in a particular arrangement in the example of FIG. 4, the cable securing interface 460 of the clip 450 can include any suitable type of cable-securing interface, such as a clasp, a hook, etc.

FIG. 5 illustrates a topside isometric view of another example surgical tray assembly 500 having apertures 520 formed therein, according to embodiments described herein. The apertures 520 include individual holes formed in tray 502 that are each configured to receive a clip 650. In certain embodiments, the apertures 520 are arranged along a perimeter 524 of an instrument surface 512 of the tray 502. The apertures 520 are shown in FIG. 5 as spanning the entire perimeter 524 of the instrument surface 512, and along all four sides of the tray 502, with uniform spacing between adjacent apertures 520. However, the apertures 520 can also be arranged with a non-uniform spacing, and/or along only a portion of the perimeter 524. In certain embodiments, the apertures 520 are only arranged at the corners 526 of the tray 502. In certain embodiments, the apertures 520 are arranged away from the corners 526 of the tray 502. Additionally, the apertures 520 shown in FIG. 5 are shown as being formed in the instrument surface 512 of the tray 502, however, in certain embodiments, the apertures 520 can be formed in side walls of the tray 502 or in a bottom surface of the tray 502.

The apertures 520 can have any suitable geometry for receiving clips 650. In certain embodiments, the apertures 520 include circular or cylindrical openings extending into the tray 502. In such embodiments, the apertures 520 can include threaded walls configured to mate with corresponding threads on the clips 650, thereby provided a threaded coupling mechanism for attaching the clips 650 to the tray 502. However, other geometries and types of coupling mechanisms are further contemplated for the apertures 520. For example, in certain embodiments, the apertures 520 include walls having snap fit features, such as protrusions or cavities, configured to snap fit with corresponding features on the clips 650. In certain embodiments, the apertures 520 are shaped and sized to facilitate an interference fit or press fit with the clips 650. Various clips 650 are shown attached to apertures 520 in FIG. 5 for reference.

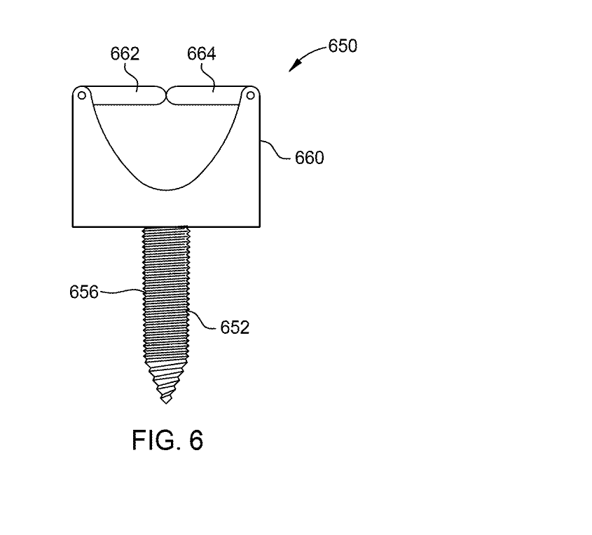

FIG. 6 illustrates an example clip 650 configured to be releasably attached in one of the apertures 520 for securing one or more cables (e.g., instrument cables) to the surgical tray 502. Similar to FIG. 4, the clip 650 includes a base 652 and a cable securing interface 660 comprising a first retaining arm 662 and a second retaining arm 664. Generally, the retaining arms 662 and 664 of the cable securing interface 660 can function similar to the retaining arms 464 and 466 described above. The primary difference between clip 650 and 450 is in the mechanism of attachment to the respective tray.

In certain embodiments, the base 652 of clip 650 can include one or more retention features configured to engage with corresponding retention features within the apertures 520. In certain examples, the retention features include snap fit features, such as protrusions or cavities, configured to mate with corresponding snap fit features formed in sidewalls of the apertures 520. In certain embodiments, the retention features include one or more threads 656, as shown in FIG. 6, configured to mate with corresponding threaded walls of the apertures 520, thereby providing a threaded coupling mechanism for attaching the clip 650 within the aperture 520. In still other embodiments, the base 652 is shaped and/or sized to form an interference fit or includes one or more retention features configured to press fit with the apertures 520.

In embodiments where the clip 650 is configured to snap fit, interference fit, or press fit into an aperture 520, the clip 650 can be inserted into the aperture 520 by a user pressing down the clip 650 with sufficient force to engage the snap fit or press fit features of the clip 650 and the aperture 520, or create an interference fit therebetween. To remove the clip 650, the user can simply pull on the clip 650 with force sufficient to overcome the engagement of the snap fit or press fit features, or overcome the interference fit. In embodiments where the clip 650 and apertures 520 are threaded, the user can fasten the clip 650 into the aperture 520 by screwing in the base 652 via rotation thereof in a first direction and can remove the clip 650 via rotation thereof in a second direction.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to "one embodiment", "an embodiment", or similar language means that a particular feature, structure, or characteristic described in connection with the indicated embodiment is included in at least one embodiment of the present disclosure. Thus, the phrases "in one embodiment", "in an embodiment", and similar language throughout this specification may, but do not necessarily, all refer to the same embodiment.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments may fall within the scope of the appended claims.

What is claimed is:

1. A surgical tray system, the surgical tray system comprising:
a tray comprising a track positioned on an instrument surface of the tray; and
a clip configured to:
be adjustably coupled to and positioned along the track, and
couple to a cable of a surgical instrument and secure the cable on the tray.

2. The surgical tray system of claim 1, wherein the track comprises at least one L-track comprising a linear channel with an aperture along the linear channel, and wherein the aperture is configured to receive and secure the clip to the at least one L-track.

3. The surgical tray system of claim 1, wherein the track comprises at least one T-track comprising a linear channel, wherein the linear channel is configured to receive and secure the clip to the at least one T-track.

4. The surgical tray system of claim 1, wherein the track comprises at least one E-track comprising a singular rail having a slot formed along a length of the E-track, wherein the slot is configured to receive and secure the clip to the at least one E-track.

5. The surgical tray system of claim 1, wherein the clip comprises a spring-biased clip configured to be secured within the track.

6. The surgical tray system of claim 1, wherein the clip comprises a cable securing interface configured to secure the cable of the surgical instrument on the tray, wherein the cable securing interface is a U-shaped structure having two retaining arms configured to receive the cable.

7. The surgical tray system of claim 6, wherein the clip further comprises a stud configured to interact with a lip of the track and secure the clip within the track.

8. The surgical tray system of claim 1, wherein the track is positioned along a perimeter of the instrument surface of the tray via interference fastening, mechanical interlocking joints, welding, or adhesives.

9. The surgical tray system of claim 1, wherein the track is molded into a perimeter of the instrument surface of the tray.

10. A surgical tray system, the surgical tray system comprising:
a tray comprising an aperture positioned on an instrument surface of the tray; and
a clip configured to:
be positioned within the aperture, and
couple to a cable of a surgical instrument and secure the cable on the tray.

11. The surgical tray system of claim 10, wherein the aperture comprises a hole within the instrument surface of the tray.

12. The surgical tray system of claim 10, wherein the clip comprises a base configured to be screwed, snap fit, interference fit, or press fit into the aperture to secure the clip within the aperture.

13. The surgical tray system of claim 12, wherein the base of the clip comprises a threaded base and the aperture is a threaded hole.

14. The surgical tray system of claim 12, wherein the base of the clip comprises a protrusion and the aperture comprises a cavity configured to retain the protrusion of the base of the clip.

15. The surgical tray system of claim 12, wherein the clip further comprises a cable securing interface configured to secure the cable of the surgical instrument on the tray, wherein the cable securing interface is a U-shaped structure having two retaining arms configured to receive the cable.
